(19) United States
(12) Patent Application Publication
(10) Pub. No.: US 2011/0143432 A1
(43) Pub. Date: Jun. 16, 2011

(54) USE OF EXTRACT OF SELENIUM-ENRICHED YEAST (SE-YE) IN MAMMALIAN CELL CULTURE MEDIA FORMULATIONS

(75) Inventors: Monika Drews, Tallinn (EE); Reet Rumvolt, Harku vald (EE); Karoli Voodla, Tallinn (EE)

(73) Assignees: AS Vahiuuringute Tehnoloogia Arenduskekus, Tallinn (EE); Celecure AS, Tallinn (EE); Tallinn University of Technology, Tallinn (EE); Pohja-Eesti Regionaalhaigla SA, Tallinn (EE); Inbio OU, Tallinn (EE); Kevelt AS, Talinn (EE); Cambrex Tallinn AS, Tallinn (EE)

(21) Appl. No.: 12/997,058

(22) PCT Filed: Jun. 9, 2009
(86) PCT No.: PCT/EE2009/000010

§ 371 Date: Mar. 3, 2011

Related U.S. Application Data
(60) Provisional application No. 61/059,874, filed on Jun. 9, 2008.

(51) Int. Cl. C12N 5/16; C12N 5/071

(52) U.S. Cl. 435/326; 435/404; 435/358

(57) ABSTRACT

The invention relates to the use of extract of selenium-enriched yeast (Se-YE) as a supplement to the serum-free cell culture media formulations. The cell culture media comprising this supplement are particularly suitable for cultivating mammalian cells and for production of recombinant proteins and monoclonal antibodies.

### TECHNICAL FIELD

This invention relates to the field of mammalian cell and tissue culture media. More specifically, the present invention relates to in vitro cultivation of mammalian cells, especially in serum-free culture media formulations supplemented with yeast extract containing organic selenium (extract of selenium-enriched yeasts-selenium yeast extract—Se-YE). The use of this invention is particularly suited for cultivating of mammalian cells and for production of recombinant proteins and monoclonal antibodies including therapeutical ones with mammalian cell cultures.

### BACKGROUND ART

For cultivation of cells, particularly eucaryotic cells, and more specifically mammalian cells, there is a constant need to use special serum-free culture media with growth promoting supplement that provides the nutrient substances and growth nutrient substances that are required for efficient cultivation of the cells in vitro and especially for the production of the high quality proteins or monoclonal antibodies that are desired. For the efficient production of biological products, such as proteins or monoclonal antibodies, it is important that an optimal cell density is achieved as well as increased protein expression to obtain maximal product yield.

Cell culture media provide the nutrients necessary to maintain and grow cells in a controlled, artificial in vitro environment. The characteristics and compositions of cell culture media vary depending on the particular cellular requirements. Requirements vary in accordance with cell line, culture size and cultivation conditions. Important parameters include similarity, pH and nutrient formulations.

Media formulations have been used to cultivate a number of different cell types including animal, plant and bacterial cells. In the cultivation process cells in culture catabolize available nutrients in culture media producing useful biological substances such as monoclonal antibodies, hormones, growth factors, etc. Optimal media provide maximally long time of growth of the cells in culture and synthesis of the proteins. Mammalian cells are mostly used for production of proteins due to the correct packaging of the product. This is achieved in optimal stable controlled growth environment of the cells. Such products have therapeutic applications and, with the advent of recombinant DNA technology, cells can be engineered to produce large quantities of these products. Cultured cells are also used for the in vitro cultivation of tissues for medical transplantation. Thus, the ability to cultivate cells effectively is necessary for the production of useful substances with constant high quality which are not otherwise obtained by biologically safe and cost-effective means.

Cell culture media formulations are well documented in the literature, and a number of media formulations and serum substitutes are commercially available. In early cell culture research, media formulations were based on the chemical composition and physicochemical properties (e.g. osmolarity, pH, etc.) of blood and were referred as “physiological solutions” (Ringer, S., 1880; Waymouth, C., 1965; Waymouth, C., 1970). Typical components of cell culture media include amino acids, organic and inorganic salts, vitamins, trace metals, sugars, lipids and nucleic acids, the types and amounts of which may vary depending upon the particular requirements of given cell or tissue type. However, cells in different tissues of the mammalian body are exposed to different microenvironments with respect to oxygen/carbon dioxide partial pressure and concentrations of nutrients, vitamins and trace elements. Accordingly, successful in vitro culture of different types of cells often requires the use of different media formulations. In vitro cultivation of mammalian cells in suspension cultures (including flask, spinner and bioreactor cultures) results extensive physical stress for cells coursed by agitating whereas foaming should be avoided. The effective scale-up of the cultivation process is achievable only in serum-free suspension culture of mammalian cells. Therefore, the key issue in improvement production of recombinant proteins or monoclonal antibodies as drug candidates is the development and optimization of economical and effective serum-free media suitable for scale-up of a suspension of mammalian cell serum-free cultures.

Typically cell culture media formulations are supplemented with a range of additives, including undefined components like fetal bovine serum (FBS) (5-20% v/v), several animal-derived proteins and/or protein hydrolysates of bovine origin. While FBS is the most commonly applied supplement in animal cell culture media, other serum sources are also routinely used, including newborn calf, horse and human. These types of chemically undefined supplements serve several useful functions in cell culture media (Lambert, K. J. et al., 1985). For example, these supplements provide carriers or chelators for labile or water-insoluble nutrients; bind and neutralize toxic moieties; provide hormones and growth factors, protect inhibitors and essential, often undefined or unidentified low molecular weight nutrients; also protect cells from physical stress and damage. Thus, serum and/or animal extracts are commonly used as supplements to provide an optimal culture medium for the cultivation of animal cells.

In general, serum or serum-derived substances such as albumin, transferrin or insulin, may contain unwanted agents that can contaminate the cell cultures and the biological products obtained therefore. The use of serum or animal extracts in cell culture applications has several drawbacks (Lambert, K. J. et al., 1985). For example, the chemical composition of those supplements may vary between lots, even from the single supplier. They may also be contaminated with infectious agents (e.g., prions, mycoplasma, and viruses) who can seriously undermine the health or the cultured cells when these contaminated supplements are used in cell culture media formulations and may additionally pose a health risk in cell therapy and other clinical applications. Moreover, bovine serum and products derived thereof bear the risk of the presence of prions causing spongiform encephalopathy (BSE) in humans or animals. In addition, all serum-derived products can be contaminated by unknown constituents. Cell surface chemistry, which is a critical portion of the in vitro microenvironment for many cell types, can be adversely modified via absorption or incorporation of serum or extract of proteins. The use of undefined components such as serum or animal cell extracts also prevents the true definition and elucidation of the nutritional and hormonal requirements of the cultured cells, thus eliminating the ability to study, in a controlled way, the effect of specific growth factors or nutrients on cell growth and differentiation in culture. Moreover, undefined supplements prevent the researcher from studying aberrant growth and differentiation and the disease-related changes in cultured cells. Serum and animal extract supplements of culture media can also complicate and increase the costs of growth of cells and purification of the desired substances from the culture media due to non-specific co-purification of serum or extract proteins. Serum in spinner or bioreactor process causes foaming, which physically damages cultivated cells.

As the market of biopharmaceuticals rapidly grows, the biotechnological industry of biological substances, particularly in the field of expression of therapeutical proteins and their manufacturing methods are under increasing regulatory security. Especial attention is paid if the cells are used for production of medicinal agents or vaccines for human administration. The use of animal sourced components (serum, animal extracts) in the culture media used for production of therapeutic proteins in mammalian cell culture for expression of therapeutic proteins is currently not acceptable (EMEA, 2002).

Therefore, many attempts have been made to provide efficient host systems and cultivation conditions that do not require serum or other animal protein compounds. To prevent the drawbacks of the use of serum or animal-sourced components, a number of serum-free media have been developed. Simple serum-free media typically includes basal medium, vitamins, amino acids, organic or inorganic salts, and optionally additional components to make the medium nutritionally complex. These media, which are often specifically formulated to support the culture of a single cell type, incorporate defined quantities of purified growth factors, lipoproteins and other proteins usually provided by the serum or extract supplement. Since the components (and concentrations) of such culture media are precisely known, these media are generally referred to as “defined culture media” and often as “serum-free media” or “SFM”. A number of SFM formulations are commercially available.

SFM generally provide several distinct advantages to the user. For example, the use of SFM facilitates the investigation of the effects of a specific growth factor or other medium component on cellular physiology, which may be masked when the cells are cultivated in serum- or extract-containing media. In addition, SFM typically contain much lower quantities of protein (SFM are often called “low protein media”) than those containing serum or extracts, rendering purification or biological substances produced by cells cultured in SFM far simpler and more cost-effective. Some extremely simple SFMs, which consist essentially of vitamins, amino acids, organic and inorganic salts and buffers have been used for cell culture. Such media (often called “basal media”), however are usually seriously deficient in the nutritional content required by most animal cells. Accordingly, most SFM incorporate additional components into basal media to take the media more nutritionally complex, while maintaining the serum-free and low protein content. Examples of such components include serum albumin from bovine (BSA), lipids, insulin etc. derived from natural or recombinant sources.

However, existing serum-free media still do not provide efficient growth promoting effect, maximal viable cell culture densities and cell culture viabilities required nowadays for growing needs of production of biologicals including therapeutical proteins with mammalian cell cultures. The reason is the missing of substances in the serum-free media enabling necessary efficiency of the above processes.

For increasing the nutritional abilities of the serum-free media animal-derived supplements and non-animal derived ones are used.

Animal-derived medium supplements are still commonly used. The use of animal-derived supplements in cell culture media, however, has certain drawbacks. For example, there is a risk that the culture medium and/or products purified from it may be immunogenic, particularly if the supplements are derived from an animal different from the source of the cells to be cultured. Thus, if biological substances to be used as therapeutics are purified from such culture media, certain amounts of these immunogenic proteins or peptides may be co-purified and may induce an immunological reaction, including anaphylaxis, in an animal receiving such therapeutics.

To avoid the use of animal protein supplement in serum-free cell culture medium, several attempts have been made to make animal cell culture media that are completely free of animal proteins. For example, in some approaches hydrolysates of plants (soy, wheat, barley, rye, oats etc.) have been used (Franek et al., 2002; US2004/0077086; US2006/0286668; WO99/57246; WO2004/0171152; WO/2004/005493; U.S. Pat. No. 6,406,909; US2008/0064080).

The use of wheat hydrolysate is likely to be quite unfavourable for the culture of many animal cells and tissues, since wheat peptides are known to be toxic or to induce toxic effects in vitro and in vivo, particularly in the cells and tissues of the gastrointestinal systems of some mammals, including humans (Strober, W., et al., Ann. Int. Med. 83:242-256 (1975); Auricchio, S., et al., Pediatr. Res. 22(6):703-707 (1987). Moreover, extracts from certain plants including wheat, barley, rye and oats have been shown to inhibit protein synthesis in cell-free systems derived from animal cells (Coleman, W. H., and Roberts, W. K., Biochim. Biophys. Acta 696: 239-244 (1987) suggesting that the use of plant hydrolysates from these plants in mammalian cell culture media may inhibit, rather than stimulate, the growth of animal cells in vitro.

Another non-animal growth promoting supplement which has been incorporated in some mammalian cell culture media is yeast extract (Sung et al., 2003). Yeast extract is a product of autolysis of baker yeast cells. The exact chemical compounds of yeast extract responsible for growth promotion in mammalian cell cultures are still not known.

Vitamines, purines, pyrimidines (Wu, 1998), amino acids and lipids are unlikely involved in growth promotion. One possible explanation for growth promotion could be that peptides included in yeast extract act as growth factors or hormones. Taking into account that even a fragment (an oligopeptide) of growth factor can act as a complete growth factor (Azuma, 1989), it is possible that small peptides of yeast extract have this function. Yeast extract (hydrolysate) supplement is used to replace serum in insect cell culture (Maiorella, 1988) or in serum-free media for mammalian cell cultures, especially for developing animal component-free media for mammalian producer cell lines (Sung et al., 2003; Drews and Hunt, 2007). Yeast extract is also a key component for achieving a high density cell cultures as described in scientific papers (Drews et al., 1998; Drews et al., 2000; Drews et al., 2003; Kasemets et al., 2003; Paalme et al. 2006).

The use of conventional yeast extract (hydrolysate) in serum-free mammalian cell cultivation in vitro is described in some patents (WO99/57246, US2006/0286668 and US2004/0171152; U.S. Pat. No. 6,406,909, WO2004/005493 and US2008/0064080; WO96/15231; US2004/0077086; WO98/15614; WO00/03000).

However, as prove the experiments conducted by the inventors of the present invention, the conventional yeast extract alone does not allow achieving high enough maximal viable cell densities and maximal culture viabilities needed for extensive cell cultivation and in additional stress conditions of intensive protein synthesis with mammalian cell cultures.

### Selenium-Yeast Extract

Inorganic selenium is a well-known trace element for cell growth and development, and its positive role in biological system includes detoxification of free radicals by activating glutathione peroxidase (Zhang et al., 2006). In cell culture, selenium can help cells to detoxify the medium thus protecting them from oxidative damage. Novel function of selenium is a highly effective carrier to deliver iron for cell metabolism and respiration and therefore the iron-transporter function of bovine serum transferrin should be replaced in serum-free medium (Zhang et al., 2006). As mammalian cells do not have active biochemical pathways for incorporating selenium in inorganic form (as selenite for example) into sulphur-containing amino acids (Kyoto Encyclopedia of Genes and Genomes), selenium in its organic form is necessary for cell functioning (Kajander et al., 1990; Zeng, 2002). However, bakers yeast (Saccharomyces cerevisiae) is able to utilize and transform inorganic selenium to organic form. During their metabolism selenite is transformed first to selenocystein and further to selenomethionine. Selenium-enriched yeasts are able to accumulate ca 3000 μg/g selenium, of which about 90% is in the form of selenomethionine. Selenomethionine is much more utilizable by mammalian cells (Bansal and Kaur, 2002) and at higher concentrations less toxic to them than an inorganic selenium.

Therefore, mammalian cell culture medium supplement such as selenium yeast extract where organic form of selenium and effect of yeast extract are synergistically combined has profound positive effect on mammalian cells growth in vitro.

So far, only inorganic selenium (selenite) and/or conventional yeast extract has been used in serum-free media.

In selenium-enriched yeast biomass, selenium is organically bound into proteins mostly via two selenium-containing amino acid—selenocystein (about 70%) and selenomethionine (about 10%). Main selenoproteins in cells are enzymes with antioxidative properties like glutathione peroxidase and thioreductase, but also for example selenophosphate synthetase (Behne and Kyriakopoulos, 2001; Gladyshev and Hatfield, 1999). WO2006/103350 describes the preparation of Se-YE and its antioxidant properties and its possible use as antioxidant additive, in food for humans and animals or in cosmetics.

However, selenium yeast extract has not been used in media for mammalian cell cultures. According to our knowledge there is no literature data available about the use of Se-YE in cultivation of mammalian cells and particularly in vitro serum-free suspension cultures.

Therefore, a current need exists to increase the yield of recombinant protein and monoclonal antibodies by enhancing specific growth rate, optimal metabolic activity, maximal viable cell density and maximal culture viability. The present invention provides selenium yeast extract as a medium supplement for in vitro cultivation of mammalian cells.

### SUMMARY OF THE INVENTION

Resulting from the above, the objective of the present invention is to provide the supplement to the serum-free medium, which essentially increases the nutritional abilities of the serum-free medium, thus enabling to achieve higher maximal viable cell densities and higher maximal culture viabilities of the cultivated mammalian cells.

For achieving of the above objective, the extract of selenium enriched yeast (Se-YE) is used as a supplement to the serum-free mammalian cell culture media formulations provided for in vitro cultivation of mammalian cells.

The mammalian cells to be cultivated are 5E1/H3 hybridoma cells (produce murine monoclonal antibodies against human transcription factor Gli3) and Chinese Hamster Ovary (CHO) cells.

The methods for cultivation of mammalian cells are selected from batch, fed-batch, chemostat, turbidostat, accelerostat type performed in flasks, spinners or bioreactors.

The selenium enriched yeast comprises organic form of selenium.

### BRIEF DESCRIPTION OF THE DRAWINGS

FIG. 1 shows the effect of extract of selenium-enriched yeast on the growth of 5E1/H3 hybridoma cells in serum-free complete medium in T-flasks.

FIG. 2 shows the effect of optimal concentration of extract of selenium-enriched yeast on the growth of 5E1/H3 hybridoma cells in serum-free complete medium in spinner flasks.

FIG. 3 shows the effect of extract of selenium-enriched yeast on monoclonal antibody production produced by 5E1/H3 hybridoma cells in serum-free complete medium.

FIG. 4 shows the effect of extract of selenium-enriched yeast on the growth of CHO cells in serum-free complete medium in shaker flasks.

### DISCLOSURE OF THE INVENTION

### Definitions

In the following description, a number of terms conventionally used in the field of cell culture media are used extensively. In order to provide a clear and consistent understanding of the specification and claims and the scope of the invention the following definitions are provided.

The phrase “mammalian cell culture” or “cell culture” refers to the maintenance of cells in an artificial, in vitro environment.

The phrases “cell culture medium”, “culture medium” (plural “media”) and “medium formulation” refer to a nutritive solution for cultivating cells.

The term “cultivation” refers to the maintenance of cells in vitro under conditions favoring growth and continued viability of the cells.

The term “culture viability” refers to the ratio of viable cells in culture to all the cells (viable plus non-viable) in the culture and is expressed in percentages.

The terms “T-flask, spinner flask, bioreactor” refers to different culture vessels that can provide an aseptic environment for culturing the cells.

The terms “batch, fed-batch, turbidostat, chemostat, accelerostat” refers to different cultivation methods of cells.

The term “extract” refers to a composition comprising a concentrated preparation of the components of a substance (selenium-enriched yeast), typically formed by the treatment of the substance by hydrolysis or enzymatic actions (autolysis).

The term “extract of “selenium-enriched yeast (Se-YE)” refers to extract made by autolysis from selenium-enriched yeast biomass. The short form of term “selenium-enriched yeast (Se-YE)” is Se-YE.

The terms “component”, “nutrient”, “ingredient”, “medium supplement” refer to any compound whether of chemical or biological origin, that can be used in cell culture media to maintain or promote the growth or proliferation of cells and production of biologicals such as recombinant proteins and monoclonal antibodies by the cells. These terms can be used interchangeable and are all meant to refer to such compounds.

The term “non-animal origin” or “derived from non-animal sources” refers to the origin of the component. Such non-animal sources include bacteria, yeasts, fungi and plants.

The term “basal medium” refers to the cell culture medium that is aqueous-based and comprises a number of ingredients in a solution of deionized, distilled water. Any basal medium can be used in accordance with the present invention. The basal media usually include following ingredients: amino acids, vitamins, organic and/or inorganic salts, trace elements, buffering salts, sugars and other components. The basal media may be obtained commercially, for example from Sigma.

The term “complete medium” refers to basal medium, which is supplemented with additional medium components that are needed for cells growth promotion or continued maintenance. For example, such components are insulin, albumin, lipids, Pluronic-F68, ferric citrate, inorganic selenite etc.

Complete medium forms when the basal medium and the above-mentioned additional components are mixed together in solution.

For experiments described in the present invention two serum-free complete media: one for 5E1/H3 hybridoma and the other for CHO cells were composed. For composing serum-free complete medium for 5E1/H3 hybridoma cells (see Table 1), yeast extract (Sigma product Y4375), albumin (Sigma product nr. A 8806), Pluronic F-68 (Sigma product nr. P-1300), ferric-citrate (Sigma product nr. F 3388), insulin (Sigma product nr. 1-6634) and HEPES (Amresco nr. 0485) were added into commercial basal medium (Sigma product D0547). For composing serum-free complete medium for CHO cells (see Table 2), yeast extract (Sigma product Y4375), albumin (Sigma product nr. A 8806), Pluronic F-68 (Sigma product nr. P-1300), ferric-citrate (Sigma product nr. F 3388), insulin (Sigma product nr. I-6634), HEPES (Amresco nr. 0485), HT supplement (Gibco nr. 41065-012) and penicillin-streptomycin (Gibco nr. 15070-063) were added into commercial basal medium (Gibco product nr. 31331).

In the experiments of the present invention the serum-free complete media for 5E1/H3 and CHO cells were supplemented at different concentrations of extract of selenium-enriched yeast.

Extract of selenium-enriched yeast (Se-YE) was made in-house from selenium-enriched yeast (Saccharomyces cerevisiae) biomass which was produced in-house by fed-batch cultivation strategy using inorganic selenite in feeding medium as described in Külvet, 2003. Se-YE was produced by autolysis from produced selenium-enriched yeast biomass as described by Suphantharika, 1997. For autolysis washed selenium-enriched yeast biomass (dry matter content 20%, selenium concentration 2135 ppm per dry matter) was kept 48 h, at 50° C. (pH=5.5) with slight agitation in fermenter. Cell debris was removed by centrifugation at 10000 rpm and remained extract (dry matter 9.5%) was filter-sterilized and used as a medium supplement in experiments of the present invention.

This medium supplement was added to complete cell culture media of mammalian cells (5E1/H3, CHO).

The optimal concentration ranges of extract of selenium-enriched yeast (Se-YE) for different cell lines and different basal and complete media are different. The optimal concentration range of supplemented extract of selenium-enriched yeast for 5E1/H3 hybridoma and CHO cells was found in several series of experiments.

Optimal concentration range found in the present invention for 5E1/H3 hybridoma cells was between 0.05-0.1 g/L and for CHO cells 0.05-0.5 g/L.

Cells, which can be cultivated in media with above mentioned growth supplement, are those of animal origin, especially cells obtained from mammals. Mammalian cell lines particularly suitable for cultivation in the medium with mentioned growth supplement include especially 5E1/H3 hybridoma and CHO cells and mammalian cells similar to those.

### MODES FOR CARRYING OUT THE INVENTION

### Materials and Methods

5E1/H3 hybridoma cells (Hunt et al., 2007) and CHO cells (Gibco) were cultivated in T-flasks (10 ml), shaker flasks (10 ml) or in spinner flasks (Techne) (50 ml) at 37° C. in CO₂ (5%) incubator. 5E1/H3 cells were routinely cultivated on serum-free complete medium for hybridoma cells given in Table 1. CHO cells were routinely cultivated on serum-free complete medium for CHO cells given in Table 2. To study the effect of Se-YE on growth of 5E1/H3 and CHO cells, Se-YE at different concentrations was added to above-mentioned complete serum-free media. Viable cell density was measured by hemocytometer and culture viability determined by Trypan Blue exclusion test. Monoclonal antibody (MAb) production by hybridomas was determined by enzyme linked immunosorbent assay (ELISA).

TABLE 1
Composition of serum-free complete
medium for 5E1/H3 hybridoma cells

| | Concentration | | Company |
|---|---|---|---|
| Component | | | |
| Basal medium DMEM/F12 | According to | | Sigma Product nr. D0547 |
| (1:1), with 1-glutamine, | producer's | | |
| trace elements, without | protocol | | |
| HEPES and | | | |
| sodium bicarbonate | | | |
| Additional components | | | |
| Yeast extract | 5 | g/L | Sigma Product nr. Y 4375 |
| ultrafiltrate | | | |
| Albumin | 3 | g/L | Sigma Product nr. A 8806 |
| Pluronic-F-68 | 1 | g/L | Sigma Product nr. P-1300 |
| Ferric-citrat FeC₆H₅O₇ | 0.147 | g/L | Sigma Product nr. F 3388 |
| Insulin | 5 | mg/L | Sigma Product nr. I-6634 |
| HEPES | 3.5745 | g/L | Amresco nr. 0485 |

TABLE 2
Composition of serum-free complete medium for CHO cells

| | Concentration | | Company |
|---|---|---|---|
| Component | | | |
| Basal medium DMEM/F12 | According to | | Gibco Product nr. 31331 |
| (1:1) + GlutaMax | producer's | | |
| | product | | |
| Additional components | | | |
| Yeast extract | 5 | g/L | Sigma Product nr. Y 4375 |
| ultrafiltrate | | | |
| Albumin | 3 | g/L | Sigma Product nr. A 8806 |
| Pluronic-F-68 | 1 | g/L | Sigma Product nr. P-1300 |
| Ferric-citrat FeC₆H₅O₇ | 0.147 | g/L | Sigma Product nr. F 3388 |
| Insulin | 5 | mg/L | Sigma Product nr. I-6634 |
| HEPES | 3.5745 | g/L | Amresco nr. 0485 |
| HT supplement 50X | 20 | ml/L | Gibco cat. nr. 41065-012 |
| Penicillin-streptomycin | 10 | ml/L | Gibco cat. nr. 15070-063 |

The Effect of Supplementation of Serum-Free Complete Media (as Given in Table 1 and 2) with Extract of Selenium-Enriched Yeast

To study the effect of extract of selenium-enriched yeast (Se-YE) as a supplement in serum-free complete media for 5E1/H3 hybridoma cells and CHO cells, in-house made Se-YE was supplemented into complete medium of 5E1/H3 (Table 1) and CHO cells (Table 2) at different concentrations. These media were then used to examine growth, viable cell density and culture viability of 5E1/H3 hybridoma and CHO cells. The determined growth parameters (growth curves, maximal viable cell densities, maximal culture viabilities) were compared to those obtained in complete media without supplementation of Se-YE. All experiments were made in duplicates.

TABLE 3
The effect of Se-YE on maximal viable cell density and maximal
culture viability of 5E1/H3 hybridoma cells in serum-free complete
medium (Table 1) in T-flask and spinner cultures.

| Supplement | Maximal viable | |
|---|---|---|
| concentration | cell density | Maximal culture |
| (g/L) | (10⁶ cells/ml) | viability (%) |
| T-flask experiment | | |
| None | 0.69 | 92.2 |
| 0.01 | 0.86 | 95 |
| 0.05 | 0.90 | 95.3 |
| 0.1 | 1.19 | 97.3 |
| 0.2 | 0.90 | 85.5 |
| Spinner flask experiment | | |
| none | 1.53 | 96.2 |
| 0.1 | 2.04 | 97.2 |

TABLE 4
The effect of Se-YE on maximal viable cell density and
maximal culture viability of CHO cells in serum-free
complete medium (Table 2) of shaker flask cultures

| Supplement | Maximal viable | |
|---|---|---|
| concentration | cell density | Maximal culture |
| (g/L) | (10⁶ cells/ml) | viability (%) |
| None | 1.96 | 96.1 |
| 0.01 | 2.00 | 96.8 |
| 0.05 | 2.24 | 97.5 |
| 0.1 | 2.42 | 97.5 |
| 0.2 | 2.55 | 96.4 |
| 0.5 | 2.98 | 98.5 |
| 0.7 | 1.88 | 95.5 |
| 1 | 1.2 | 94.5 |
| 1.5 | 0.9 | 96.4 |
| 2.0 | 0.57 | 96.3 |

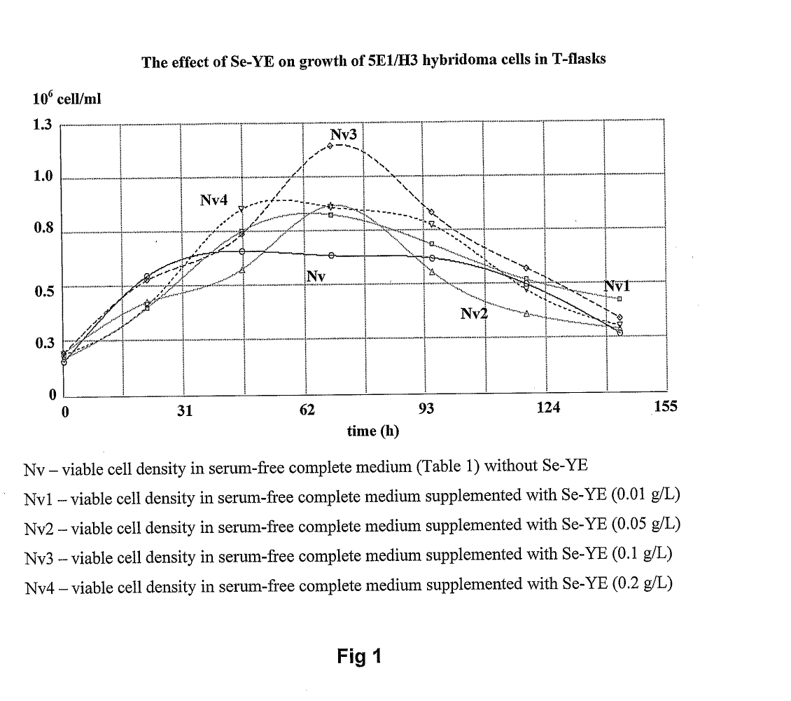

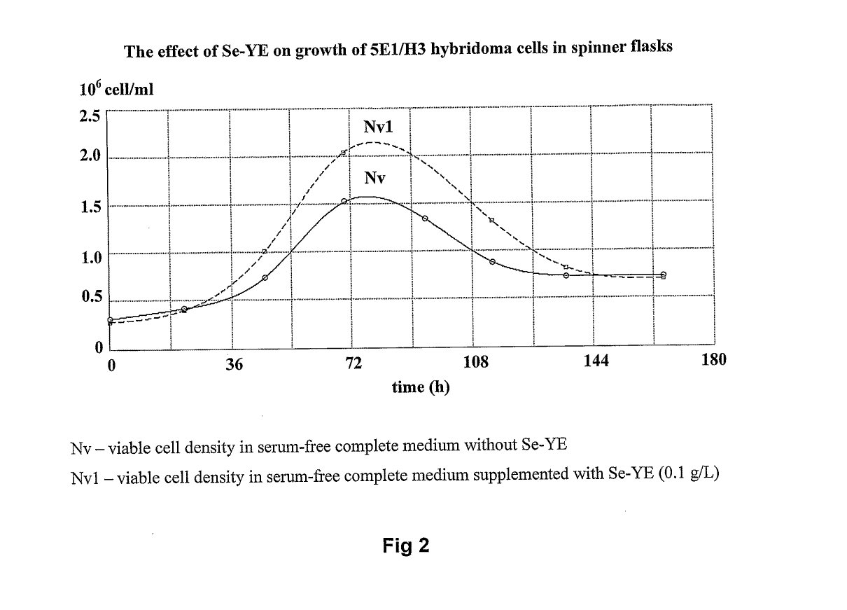

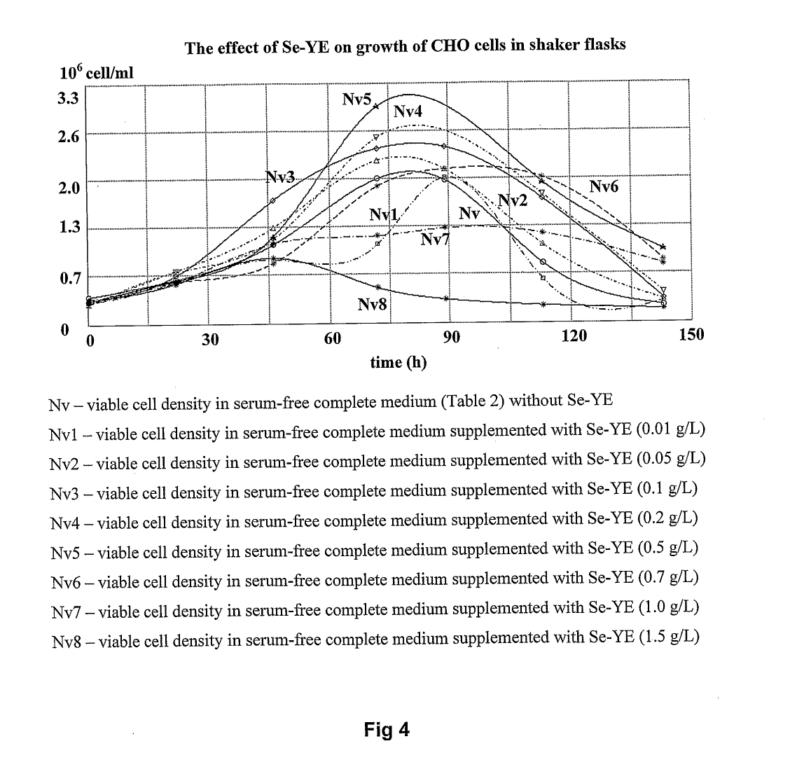

As can be seen from FIG. 1, 4 and Table 3, 4, Se-YE had a positive effect on maximal viable cell density and maximal culture viability of 5E1/H3 and CHO cells in a dose-dependent manner. The maximal effect of Se-YE was observed at 0.1 g/L for 5E1/H3 cells (FIGS. 1, 2; Table 3) in serum-free complete medium for hybridoma (Table 1) and at 0.5 g/L for CHO cells (FIG. 4, Table 4) in serum-free complete medium for CHO cells (Table 2).

### The Effect of Se-YE on Monoclonal Antibody Production in 5E1/H3 Hybridoma Cell Culture

To examine and compare the effect of Se-Ye on monoclonal antibody production three experiments with 5E1/H3 hybridoma cells were performed.

Monoclonal antibody production was measured in the three different media:

1) conventional serum-containing complete medium comprising Dulbecco's modified Eagle's Medium (DMEM) (Invitrogen) and 10% fetal calf serum;
2) serum-free complete medium as described in Table 1;
3) serum-free complete medium as described in Table 1, where yeast extract ultrafiltrate (Sigma) (5 g/L) was replaced by Se-YE (2 g/L) prepared as described above.

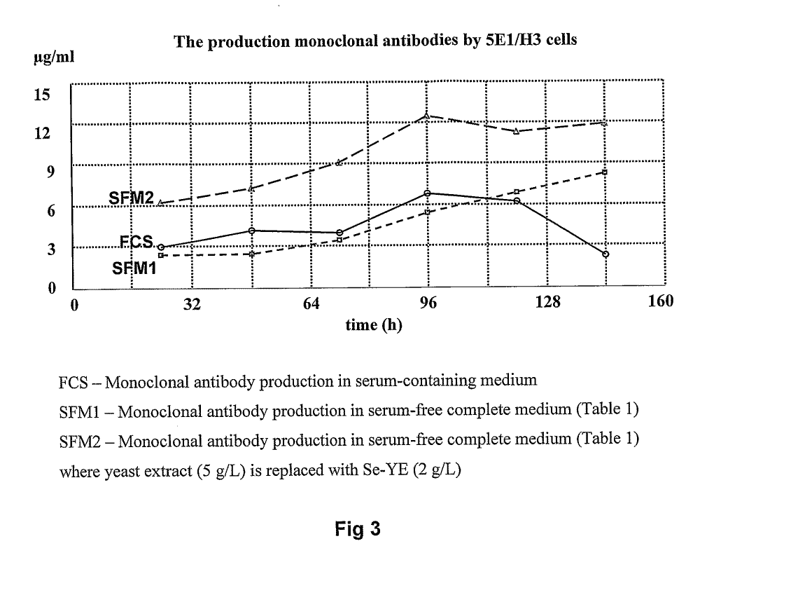

As can be seen from FIG. 3, the best productivity of monoclonal antibodies was obtained in serum-free complete medium, where yeast extract was replaced with Se-YE (SFM2).

So, the experiments conducted prove that the use of Se-YE instead of conventional extract (for example yeast extract ultrafiltrate by Sigma) in serum-free complete medium enhances the production of monoclonal antibodies by 5E1/H3 hybridoma cells.

In conclusion, supplementation of serum-free complete media with Se-YE in optimal concentrations enhances 5E1/H3 and CHO cell growth parameters such as maximal viable cell density and maximal viability of cells. At the same time, replacement of conventional yeast extract with Se-YE in serum-free complete medium affords better productivity of 5E1/H3 hybridoma cells in producing monoclonal antibodies.

### PATENT REFERENCES

US2004/0077086 A1, Animal protein free media for cultivation of cells, HELLER, EHRMAN, WHITE and MCAULIFFE
US2006/0286668 A1. Animal-cell culture media comprising non-animal or plant-derived nutrients. STERNE, KESSLER, GODSTEIN & FOX
WO99/57246 A1. Animal cell culture media comprising non-animal or plant-derived nutrients. Esmond, R. W et al.
US2004/0171152 A1. Animal cell culture media comprising non-animal or plant-derived nutrients. PRICE, GORFIEN, DANNER, PLAYSIK.
WO2004/005493 A1. Animal protein free media for cultivation of eels. REITTER, MUNDT, GRILLBERGER, KRAUS.
U.S. Pat. No. 6,406,909 B1. Serum-free medium for culturing animal cells. SHIBUYA, ATSUMI, TSUNAKAWA, NOGAKI, FLETCHER, IMADA, LYDERSEN.
US2008/0064080. Animal protein-free media for cultivation of cells. GRILLBERGER, REITER, MUNDT, DORNER.
WO2006/103350 A1. Yeast preparations with improved antioxidant properties and uses thereof. DEGRE, BAULEZ, ZHIGEN, FORBES.

### NON-PATENT REFERENCES

Azuma, N., Nagaune, S., Ishino, Y., Mori, H., Kaminogawa, S., Yamauchi, K. (1989) DNA-Synthesis Stimulating Peptides from Human □-Casein. Agric. Biol. Chem. 53 (10), 2631-2634.
Behne, D. and Kyriakopoulos, A. (2001). Mammalian selenium-containing proteins. Annual Review of Nutrition. 21: 453-473.
Drews, M., Kasemets, K., Nizamedtinov, I. and Paalme, T. (1998) Continuous cultivation of insect and yeast cells at maximum specific growth rate. Proc. Estonian Acad. Sci. Chem. 47, (4), 175-188.
Drews, M., Doverskog, M., Öhman, L., Chapman, B. E., Jacobsson, U., Kuchel, P. W. and Häggström, L. (2000) Pathways of glutamine metabolism in Spodopera frugiperda (Sf9) insect cells: evidence for the presence of the nitrogen assimilation system, and a metabolic switch by ¹H/¹⁵N NMR. J. Biotechnol. 78: 23-37.
Drews, M., Nisamedtinov, I. and Paalme T. (2003). Application of quasi-steady-state cultures. In: S. Sorvari (ed.), The 1^{st} International Congress on Bioreactor Technology in Cell-, Tissue Culture and Biomedical Applications (ISBN 952-96302-0-8), Tampere, Finland 14-18 Jul. 2003, BioBien Innovations (BBi), pp. 218-225.
Drews, M. and Hunt, R. (2007) Growth and production characteristics of four mammalian cell lines on a cost-effective serum-free medium. 20^{th} Meeting of the European Sociaty for Animal Cell Technology (ESACT), Jun. 17-20, 2007, Dresden, Germany, P-3.32.
EMEA, 2002. Note for guidance on minimizing the risk of transmitting animal spongiform encephalopathies via human and veterinary medicinal products. EMEA/410/01 Rev 2.
Gladyshev, V. N. and Hatfield, D. L. (1999) Selenocysteine-containing proteins in mammals. J. Biomed. Sci. 6:151-160
Hunt, R., Bragina, O., Drews, M., Kask, L., Timmusk, S., Valkna, A., Kogerman, P., Järveküla, L. (2007) Generation and characterization of mouse monoclonal antibody 5E1 against human transcription factor Gli3. Hybridoma, 26(3): 131-38.
Kajander, E. O., Harvima, R. J., Kauppinen, L., Akernan, L. L, Martikainen, H., Pajula, R. L. And Kärenlampi, S. O. (1990) Effects of selenomethionine on cell growth and on S-adenosylmethionine metabolism in cultured malignant cells. Biochem J. 267(3): 767-774.
Kasemets, K., Drews, M., Nisamedtinov, I., Adamberg, K. and Paalme, T. (2003)

Modification of A-stat for the characterization of microorganisms. J. of Microbiological Methods, Vol. 55(1), 187-200.
Paalme, T., Nisamedtinov, I., Abner, K., Laht, T.-M., Drews, M. and Pehk, T. (2006) Application of 13C-[2]- and 13C-[1, 2] acetate in metabolic labeling studies of yeast and insect cells. Antonie van Leeuwenhoek 89 (3-4), 443-457.
Sung, Y. H., Lim, S. W., Chung, J. Y. and Lee, G. M. (2003). Yeast hydrolysate as a low-cost additive to serum-free medium for the production of human thrombopoietin in suspension cultures of Chinese hamster ovary cells. Appl. Microbiol. Biotechnol. 63 (5): 527-536.
Zhang, J., Robinson, D. and Salmon, P. (2006) A novel function for selenium in biological system—selenite as a highly effective iron carrier for Chinese Hamster Ovary cell growth and monoclonal antibody production. Biotechnol. Bioeng. 95 (6), 1188-1197.
Zeng, H. (2002) Selenite and selenomethionine promote HL-60 cell cycle promotion. J. Nutr. 132: 674-679.
Ringer, S., (1880) J. Physiol. 3:380-393
Waymouth, C., (1965) In: Cells and Tissues in Culture, Vol. 1, Academic Press, London, pp. 99-142
Waymouth, C., (1970) In Vitro 6:109-127
Lambert, K. J. et al., (1985) In: Animal Cell Biotechnology, Vol. 1, Spier, R. E. et al., Eds., Academic Press New York, pp. 85-122.
Bansal, M. P. and Kaur, T. (2002) Growth characteristics and selenium status changes of yeast cells with inorganic and organic selenium supplementation:selenium, chemopreventive agent. J. of Medicinal Food 5 (2): 85-90.
Külvet, K. (2003) Optimization of production of selenium-enriched yeast using fed-batch and D-stat cultivation methods. Tallinn Technical University.
Maiorella, B., D., Inlow, A. Shauger & D. Harano (1988) Large-scale insect cell culture for recombinant protein production. Bio/Technology 6: 1406-1411.
Suphantharika, M., Varavinit, S. and Shobsngob, S. (1997) Determination of optimum conditions for autolyzed yeast extract production. ASEAN J. Sci. Technology Development. 14 (1), 21-28.
Wu, J. A. L., K.-I. D. (1998) Growth promotion by yeastolate and related components on insect cells. Biotechnology Techniques 12:67-70.
(Strober, W., et al., Ann. Int. Med. 83:242-256 (1975); Auricchio, S., et al., Pediatr. Res. 22(6):703-707 (1987)
Coleman, W. H., and Roberts, W. K., Biochim. Biophys. Acta 696: 239-244 (1987)

1. A use of an extract of selenium enriched yeast (Se-YE) as a supplement in a serum-free mammalian cell culture media formulations provided for in vitro cultivation of a mammalian cells.

2. The use according to claim 1, wherein the mammalian cells to be cultivated are 5E1/H3 hybridoma cells.

3. The use according to claim 1, wherein the mammalian cells to be cultivated are CHO cells.

4. The use according to claim 2, wherein a maximal viable cell density and a maximal culture viability for the 5E1/H3 hybridoma cells was achieved at a supplement concentration of 0.05-0.1 g/L.

5. The use according to claim 3, wherein a maximal viable cell density and a maximal culture viability for the CHO cells was achieved at a supplement concentration of 0.05-0.5 g/L.

6. The use according to claim 1, wherein the methods for cultivation of mammalian cells are selected from the group consisting of: batch, fed-batch, chemostat, turbidostat, accelerostat type bioreactor processes.

7. The use according to claim 1, wherein selenium enriched yeast comprises an organic form of selenium.
